(19) United States
(12) Patent Application Publication
(10) Pub. No.: US 2006/0252763 A1
(43) Pub. Date: Nov. 9, 2006

(54) Treatment of von hippel lindau disease

(76) Inventor: William Kaelin, Boston, MA (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/538,990

(22) PCT Filed: Dec. 16, 2003
(86) PCT No.: PCT/IB03/06091

§ 371 Date: Jun. 21, 2006

Related U.S. Application Data
(60) Provisional application No. 60/435,049, filed on Dec. 20, 2002.

(51) Int. Cl. A61K 31/501

(52) U.S. Cl. 514/252.02; 514/252.03

(57) ABSTRACT

The present invention relates to a method of treating VHL comprising administering a therapeutically effective amount of a compound described in the specification to a warm-blooded animal in need thereof.

The present invention relates to a method of treating a warm-blooded animal, especially a human, having the von Hippel-Lindau disease (VHL), comprising administering to said animal a therapeutically effective amount of a 4-pyridylmethyl-phthalazine derivative, especially a compound of formula I as defined herein, alone or in combination with further therapeutic measures, for example, those defined herein; the use of a 4-pyridylmethyl-phthalazine derivative for the preparation of a medicament for the treatment of VHL; and to a commercial package comprising a pharmaceutical composition together with instructions for its use in the treatment of VHL.

VHL is a genetic multi-system disorder characterized by the abnormal growth of tumors in certain parts of the body (angiomatosis). The tumors of the central nervous system are benign and are comprised of a nest of blood vessels and are called hemangioblastomas (or angiomas in the eye). Hemangioblastomas may develop in the brain, the retina of the eyes, and other areas of the nervous system. Other types of tumors develop in the adrenal glands, the kidneys, or the pancreas. Symptoms of VHL vary among patients and depend on the size and location of the tumors. Symptoms may include headaches, problems with balance and walking, dizziness, weakness of the limbs, vision problems, and high blood pressure. Cysts and/or tumors (benign or cancerous) may develop around the hemangioblastomas and cause the symptoms listed above. Individuals with VHL are also at a higher risk than normal for certain types of cancer, especially kidney cancer. VHL may result in blindness and/or permanent brain damage. Death is usually caused by complications of brain tumors or kidney cancer.

The most common clinical manifestations of VHL are retinal, cerebellar, spinal and medullary hemangioblastomas, renal cysts and carcinoma, pancreatic cysts, pheochromocytoma and papillary cystadenoma of the epididymis. If a family history of retinal or central nervous system hemangioblastoma (Hb) exists, only one Hb or visceral lesion (renal tumors, pancreatic cysts or tumors, pheochromocytoma, papillary cystadenomas of the epididymis) is required to make the diagnosis of VHL. For isolated cases without a clear family history, two or more Hbs or one Hb and a visceral manifestation is required.

Surprisingly, it was found that 4-pyridylmethyl-phthalazine derivatives are useful for the treatment of VHL.

4-Pyridylmethyl-phthalazine derivatives which a suitable for the present invention, their preparation and suitable pharmaceutical formulations containing the same are described in WO00/59509, EP02/04892, WO01/10859 and, especially, in U.S. Pat. No. 6,258,812, which are here incorporated by reference.

4-Pyridylmethyl-phthalazine derivatives and, in particular 4-pyridylmethyl-phthalazine derivatives of formula I, 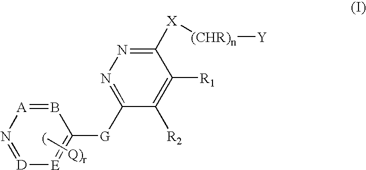
wherein the radicals and symbols have the meanings as defined below, the N-oxides of these 4-pyridylmethyl-phthalazine derivatives, as well as the salts thereof, are tyrosine kinase inhibitors, which were designed to inhibit the vascular endothelial growth factor (VEGF) signal transduction by binding directly to the ATP-binding sites of VEGF receptors. Such 4-pyridylmethyl-phthalazine derivatives reduce the microvasculature and inhibit growth of primary tumors and metastases in animal models and are useful for treating diseases associated with deregulated anglogenesis, especially neoplastic diseases (solid tumors), such as breast cancer, cancer of the colon, lung cancer, especially small cell lung cancer, and cancer of the prostate.

In particular, the present invention relates to a method of treating VHL-related hemangioblastoma comprising administering a therapeutically effective amount of a 4-pyridylmethyl-phthalazine derivative to a warm-blooded animal in need thereof.

Hence, the invention relates to a method of treating VHL and/or VHL-related hemangioblastoma comprising administering a therapeutically effective amount of a 4-pyridylmethyl-phthalazine derivative to a warm-blooded animal in need thereof, preferably of a therapeutically effective amount of a 4-pyridylmethyl-phthalazine derivative of formula 1,

wherein
r is 0 to 2,
n is 0 to 2,
m is 0 to 4,
R₁ and R₂ (i) are lower alkyl or

(ii) together form a bridge in subformula I* 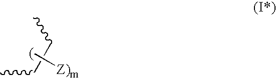
the binding being achieved via the two terminal carbon atoms, or

(iii) together form a bridge in subformula I** 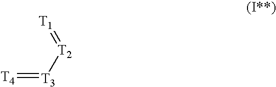
wherein one or two of the ring members T₁, T₂, T₃ and T₄ are nitrogen, and the others are in each case CH, and the binding is achieved via T₁ and T₄;
A, B, D, and E are, independently of one another, N or CH, with the stipulation that not more than 2 of these radicals are N;
G is lower alkylene, lower alkylene substituted by acyloxy or hydroxy, —CH₂—O—, —CH₂—S—, —CH₂—NH—, oxa (—O—), thia (—S—), or imino (—NH—);
Q is lower alkyl;
R is H or lower alkyl;
X is imino, oxa, or thia;
Y is unsubstituted or substituted aryl, pyridyl, or unsubstituted or substituted cycloalkyl; and
Z is amino, mono- or disubstituted amino, halogen, alkyl, substituted alkyl, hydroxy, etherified or esterified hydroxy, nitro, cyano, carboxy, esterified carboxy, alkanoyl, carbamoyl, N-mono- or N,N-disubstituted carbamoyl, amidino, guanidino, mercapto, sulfo, phenylthio, phenyl-lower alkylthio, alkylphenylthio, phenylsulfonyl, phenyl-lower alkylsulfinyl or alkylphenylsulfinyl, substituents Z being the same or different from one another if more than 1 radical Z is present;
and wherein the bonds characterized, if present, by a wavy line are either single or double bonds;
or an N-oxide of the defined compound,
or the salt of such compound having at least one salt-forming group.

The radicals and symbols as used in the definition of a compound of formula I have the meanings as disclosed in WO 98/35958 which publication is hereby incorporated into the present application by reference.

For example, 1-(4-chloroanilino)-4-(4-pyridylmethyl)phthalazine (also known as PTK787 or ZK222584), a compound of formula I, wherein r, n and m are each 0, R₁ and R² together form a bridge of subformula I*, A, B, D and E are each CH, G is methylene, X is imino, Y is 4-chlorophenyl, and the bonds characterized by a wavy line are double bonds, is most specific for KDR, but can also inhibit Fit-1 and Flt-4 and has activity against other tyrosine kinase receptors, including c-Kit.

It will be understood that in the discussion of methods, references to the active ingredients are meant to also include the pharmaceutically acceptable salts. If these active ingredients have, for example, at least one basic center, they can form acid addition salts. Corresponding acid addition salts can also be formed having, if desired, an additionally present basic center. The active ingredients having an acid group (for example COOH) can also form salts with bases. The active ingredient or a pharmaceutically acceptable salt thereof may also be used in form of a hydrate or include other solvents used for crystallization.

A preferred compound of formula I is 1-(4-chloroanilino)-4-(4-pyridylmethyl)phthalazine. More preferably, 1-(4-chloroanilino)-4-(4-pyridylmethyl)phthalazine is employed in the form of its succinate salt.

The term “VH” as used herein means VHL without pheochromocytomas as well as VHL with pheochromocytomas.

The term “treatment” as used herein comprises the treatment of patients having VHL or having the genetic disposition of said disease which treatment effects the delay of progression of the disease in said patients.

In particular, the term “hemangloblastoma” relates to CNS hemangioblastoma, especially hemangioblastoma of the brain, and/or retinal in patients hemangioblastoma with von Hippel-Lindau disease.

In a preferred embodiment of the present invention, the disease treated is refractory or not amenable to standard therapy.

In a further preferred embodiment of the present invention, the disease treated is refractory retinal hemangioblastoma that is causing impaired visual function.

For the treatment of VHL a 4-pyridylmethyl-phthalazine derivative can be administered alone or in combination with other forms of treatments, e.g. surgery or focused high-dose radiation therapy.

The person skilled in the pertinent art is fully enabled to select relevant test models to prove the hereinbefore and hereinafter mentioned beneficial effects on VHL of a 4-pyridylmethyl-phthalazine derivative. The pharmacological activity of a 4-pyridylmethyl-phthalazine derivative may, for example, be demonstrated in a suitable clinical study. Suitable clinical studies are, for example, open label non-randomized, dose escalation studies in patients with VHL alone or in combination with additional therapeutic measures, e.g., those mentioned herein. The beneficial effects on VHL can be determined directly through the results of such studies or by changes in the study design which are known as such to a person skilled in the art.

The effective dosage of a 4-pyridylmethyl-phthalazine derivative may vary depending on the particular compound or pharmaceutical composition employed, the mode of administration, the type of the VHL being treated, the severity of the VHL being treated and the co-medication. Thus, the dosage regimen of a 4-pyridylmethyl-phthalazine derivative is selected in accordance with a variety of factors including the route of administration and the renal and hepatic function of the patient. A physician, clinician or veterinarian of ordinary skill can readily determine and prescribe the effective amount of a 4-pyridylmethyl-phthalazine derivative required to prevent, counter or arrest the progress of the condition. Optimal precision in achieving concentration of the active ingredients within the range that yields efficacy without toxicity requires a regimen based on the kinetics of the active ingredients' availability to target sites.

In the present invention, 1-(4-chloroanilino)-4-(4-pyridylmethyl)phthalazine, or a pharmaceutically acceptable salt thereof, can be administered twice or more daily, for example two or three times daily, on a continuous basis, alone, or during and subsequent to other therapies in reduced amounts. A daily oral administration of an amount in the range from 300 mg to 4000 mg, for example in the range from 300 mg/day to 2000 mg/day or 300 mg/day to 1000 mg/day, in particular 300, 500, 750, 1000, 1500 or 2000 mg/day, split into two doses, is contemplated as a pharmaceutically effective amount in the twice daily regimen. A 1000 mg/day dose is given as two 500 mg doses 6 to 12 hours apart, for example about 8 hours apart, and a 2000 mg/day dose is administered as two 1000 mg doses 6 to 8 hours apart, for example about 12 hours apart.

Alternatively, the present invention embraces a treatment regimen wherein 1-(4-chloroanilino)-4-(4-pyridylmethyl)phthalazine is administered once daily at a dose in the range from 1000 mg/day to 1400 mg/day, particularly a dose of 1200 mg/day to 1300 mg/day, especially 1250 mg/day.

Moreover, the present invention provides a commercial package comprising a pharmaceutical composition together with instructions for its use in the treatment of VHL.

The present invention also relates to the use of a 4-pyridylmethyl-phthalazine derivative for the preparation of a medicament for the treatment of VHL.

1. A method of treating VHL comprising administering a therapeutically effective amount of a 4-pyridylmethyl-phthalazine derivative to a warm-blooded animal in need thereof.

2. A method of treating VHL-related hemangioblastoma comprising administering a therapeutically effective amount of a 4-pyridylmethyl-phthalazine derivative to a warm-blooded animal in need thereof.

3. Method according to claim 1 comprising administering a therapeutically effective amount of a 4-pyridylmethyl-phthalazine derivative of formula I 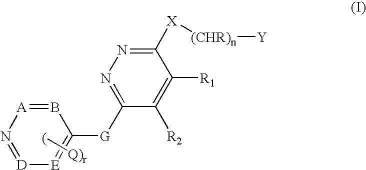
wherein
r is 0 to 2,
n is 0 to 2,
m is 0 to 4,
R₁ and R₂ (i) are lower alkyl or
(ii) together form a bridge in subformula I* 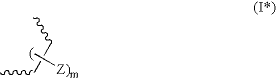
the binding being achieved via the two terminal carbon atoms, or
(iii) together form a bridge in subformula I** 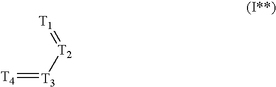
wherein one or two of the ring members T₁, T₂, T₃ and T₄ are nitrogen, and the others are in each case CH, and the binding is achieved via T₁ and T₄;
A, B, D, and E are, independently of one another, N or CH, with the stipulation that not more than 2 of these radicals are N;
G is lower alkylene, lower alkylene substituted by acyloxy or hydroxy, —CH₂—O—, —CH₂—S—, —CH₂—NH—, oxa (—O—), thia (—S—), or imino (—NH—);
Q is lower alkyl;
R is H or lower alkyl;
X is imino, oxa, or thia;
Y is unsubstituted or substituted aryl, pyridyl, or unsubstituted or substituted cycloalkyl; and
Z is amino, mono- or disubstituted amino, halogen, alkyl, substituted alkyl, hydroxy, etherified or esterified hydroxy, nitro, cyano, carboxy, esterified carboxy, alkanoyl, carbamoyl, N-mono- or N,N-disubstituted carbamoyl, amidino, guanidino, mercapto, sulfo, phenylthio, phenyl-lower alkylthio, alkylphenylthio, phenylsulfonyl, phenyl-lower alkylsulfinyl or alkylphenylsulfinyl, substituents Z being the same or different from one another if more than 1 radical Z is present;
and wherein the bonds characterized, if present, by a wavy line are either single or double bonds;
or an N-oxide of the defined compound, wherein 1 or more N atoms carry an oxygen atom,
or the salt of such compound having at least one salt-forming group,
to a warm-blooded animal in need thereof.

4. Method of claim 3 wherein the 4-pyridylmethyl-phthalazine derivative of formula I is 1-(4-chloroanilino)4-(4-pyridylmethyl)phthalazine.

5. Method according to claim 1 wherein the warm-blooded animal is a human.

6. Method according to claim 5 which comprises administering 1-(4-chloroanilino)4-(4-pyridylmethyl)phthalazine, or a pharmaceutically acceptable salt thereof, to the patient on a once daily schedule at a dose in the range from 1000 mg/day to 1400 mg/day.

7. Method according to claim 6 wherein the once daily dose is 1200 mg/day to 1300 mg/day.

8. Method according to claim 6 wherein the once daily dose is 1250 mg/day.

9. A method of treating VHL and/or VHS-related hemangioblastoma comprising administering a 4-pyridylmethyl-phthalazine derivative in an amount which is therapeutically effective against VHL to a warm-blooded animal in need thereof in combination with surgery and/or radiation therapy.

10. A commercial package comprising a 4-pyridylmethyl-phthalazine derivative together with instructions for use thereof in the treatment of VHL and/or VHS-related hemangioblastoma.

11. Use of a 4-pyridylmethyl-phthalazine derivative for the preparation of a medicament for the treatment of VHL.

12. Use according to claim 11 wherein the 4-pyridylmethyl-phthalazine derivative is 1-(4-chloroanilino)-4-(4-pyridylmethyl)phthalazine.
